SEQUENCE LISTING (19 sequences)

SEQ ID NO: 1 - DNA, 1909 bp, Homo sapiens
  mRNA (1)..(1909): This feature is the cDNA nucleic acid sequence for CD95L mRNA. CD95L is a ligand for CD95.  CD95 is APO-1/Fas and a member of the tumor necrosis factor receptor superfamily.
gaggtgtttcccttagctatggaaactctataagagagatccagcttgcctcctcttgagcagtcagcaacagggtcccgtccttgacacctcagcctctacaggactgagaagaagtaaaaccgtttgctggggctggcctgactcaccagctgccatgcagcagcccttcaattacccatatccccagatctactgggtggacagcagtgccagctctccctgggcccctccaggcacagttcttccctgtccaacctctgtgcccagaaggcctggtcaaaggaggccaccaccaccaccgccaccgccaccactaccacctccgccgccgccgccaccactgcctccactaccgctgccacccctgaagaagagagggaaccacagcacaggcctgtgtctccttgtgatgtttttcatggttctggttgccttggtaggattgggcctggggatgtttcagctcttccacctacagaaggagctggcagaactccgagagtctaccagccagatgcacacagcatcatctttggagaagcaaataggccaccccagtccaccccctgaaaaaaaggagctgaggaaagtggcccatttaacaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctctaagagaagcactttgggattctttccattatgattctttgttacaggcaccgagaatgttgtattcagtgagggtcttcttacatgcatttgaggtcaagtaagaagacatgaaccaagtggaccttgagaccacagggttcaaaatgtctgtagctcctcaactcacctaatgtttatgagccagacaaatggaggaatatgacggaagaacatagaactctgggctgccatgtgaagagggagaagcatgaaaaagcagctaccaggtgttctacactcatcttagtgcctgagagtatttaggcagattgaaaaggacaccttttaactcacctctcaaggtgggccttgctacctcaagggggactgtctttcagatacatggttgtgacctgaggatttaagggatggaaaaggaagactagaggcttgcataataagctaaagaggctgaaagaggccaatgccccactggcagcatcttcacttctaaatgcatatcctgagccatcggtgaaactaacagataagcaagagagatgttttggggactcatttcattcctaacacagcatgtgtatttccagtgcaattgtaggggtgtgtgtgtgtgtgtgtgtgtgtgtgtgtgtatgactaaagagagaatgtagatattgtgaagtacatattaggaaaatatgggttgcatttggtcaagattttgaatgcttcctgacaatcaactctaatagtgcttaaaaatcattgattgtcagctactaatgatgttttcctataatataataaatatttatgtagatgtgcatttttgtgaaatgaaaacatgtaataaaaagtatatgttaggatacaaaaaaaaaaaaaaaaaaaaaaaaaaaaaaaaaaaaaaaaaaaaaaaaaaaa

SEQ ID NO: 2 - DNA, 1888 bp, Homo sapiens
  gene (1)..(1888): This feature is the nucleic acid sequence for the CD95L gene. CD95L is a ligand for CD95.  CD95 is APO-1/Fas and a member of the tumor necrosis factor receptor superfamily.
agagagagatagagaaagagaaagacagaggtgtttcccttagctatggaaactctataagagagatccagcttgcctcctcttgagcagtcagcaacagggtcccgtccttgacacctcagcctctacaggactgagaagaagtaaaaccgtttgctggggctggcctgactcaccagctgccatgcagcagcccttcaattacccatatccccagatctactgggtggacagcagtgccagctctccctgggcccctccaggcacagttcttccctgtccaacctctgtgcccagaaggcctggtcaaaggaggccaccaccaccaccgccaccgccaccactaccacctccgccgccgccgccaccactgcctccactaccgctgccacccctgaagaagagagggaaccacagcacaggcctgtgtctccttgtgatgtttttcatggttctggttgccttggtaggattgggcctggggatgtttcagctcttccacctacagaaggagctggcagaactccgagagtctaccagccagatgcacacagcatcatctttggagaagcaaataggccaccccagtccaccccctgaaaaaaaggagctgaggaaagtggcccatttaacaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctctaagagaagcactttgggattctttccattatgattctttgttacaggcaccgagaatgttgtattcagtgagggtcttcttacatgcatttgaggtcaagtaagaagacatgaaccaagtggaccttgagaccacagggttcaaaatgtctgtagctcctcaactcacctaatgtttatgagccagacaaatggaggaatatgacggaagaacatagaactctgggctgccatgtgaagagggagaagcatgaaaaagcagctaccaggtgttctacactcatcttagtgcctgagagtatttaggcagattgaaaaggacaccttttaactcacctctcaaggtgggccttgctacctcaagggggactgtctttcagatacatggttgtgacctgaggatttaagggatggaaaaggaagactagaggcttgcataataagctaaagaggctgaaagaggccaatgccccactggcagcatcttcacttctaaatgcatatcctgagccatcggtgaaactaacagataagcaagagagatgttttggggactcatttcattcctaacacagcatgtgtatttccagtgcaattgtaggggtgtgtgtgtgtgtgtgtgtgtgtgtgtgtgtatgactaaagagagaatgtagatattgtgaagtacatattaggaaaatatgggttgcatttggtcaagattttgaatgcttcctgacaatcaactctaatagtgcttaaaaatcattgattgtcagctactaatgatgttttcctataatataataaatatttatgtagatgtgcatttttgtgaaatgaaaacatgtaataaaaagtatatgttaggatacaaat

SEQ ID NO: 3 - protein, 281 aa, Homo sapiens
  CHAIN (1)..(281): This feature is the amino acid sequence for CD95L protein.  CD95L is a ligand for CD95.  CD95 is APO-1/Fas and a member of the tumor necrosis factor receptor superfamily.
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 4 - protein, 222 aa, Mus musculus
  CHAIN (1)..(222)
  SIGNAL (1)..(20)
METDTLLLWVLLLWVPGSTGELRSVAHLTGNPHSRSIPLEWEDTYGTALISGVKYKKGGLVINETGLYFVYSKVYFRGQSCNNQPLNHKVYMRNSKYPEDLVLMEEKRLNYCTTGQIWAHSSYLGAVFNLTSADHLYVNISQLSLINFEESKTFFGLYKLGSSGSSGSSGSGYIPEAPRDGQAYVRKDGEWVLLSTFLSGPSSSSSHHHHHHSAWSHPQFEK

SEQ ID NO: 5 - DNA, 20 bp, Artificial Sequence
  oligonucleotide PCR primer optimized for amplification of Mus musculus DNA
tgatggtttgggaagtttga

SEQ ID NO: 6 - DNA, 20 bp, Artificial Sequence
  oligonucleotide PCR primer optimized for amplification of Mus musculus DNA
gtgcgtgagctctggataaa

SEQ ID NO: 7 - DNA, 20 bp, Artificial Sequence
  oligonucleotide PCR primer optimized for amplification of Mus musculus DNA
cttgcagacacctggaagaa

SEQ ID NO: 8 - DNA, 20 bp, Artificial Sequence
  oligonucleotide PCR primer optimized for amplification of Mus musculus DNA
cctctggtatcccaaggaaa

SEQ ID NO: 9 - DNA, 20 bp, Artificial Sequence
  oligonucleotide PCR primer optimized for amplification of Mus musculus DNA
cagaatgggaactgttgtgg

SEQ ID NO: 10 - DNA, 20 bp, Artificial Sequence
  oligonucleotide PCR primer optimized for amplification of Mus musculus DNA
ctccttgaggcacactttga

SEQ ID NO: 11 - DNA, 20 bp, Artificial Sequence
  oligonucleotide PCR primer optimized for amplification of Mus musculus DNA
tggttactctcctgggttcc

SEQ ID NO: 12 - DNA, 20 bp, Artificial Sequence
  oligonucleotide PCR primer optimized for amplification of Mus musculus DNA
ttcactccctgagtcacagc

SEQ ID NO: 13 - DNA, 20 bp, Artificial Sequence
  oligonucleotide PCR primer optimized for amplification of Mus musculus DNA
gagccggctgacaatacaat

SEQ ID NO: 14 - DNA, 20 bp, Artificial Sequence
  oligonucleotide PCR primer optimized for amplification of Mus musculus DNA
ctcaggttcttcctggagca

SEQ ID NO: 15 - DNA, 20 bp, Artificial Sequence
  oligonucleotide PCR primer optimized for amplification of Mus musculus DNA
attcaacggcacagtcaagg

SEQ ID NO: 16 - DNA, 20 bp, Artificial Sequence
  oligonucleotide PCR primer optimized for amplification of Mus musculus DNA
tggatgcagggatgatgttc

SEQ ID NO: 17 - protein, 11 aa, Homo sapiens
  MISC_FEATURE (1)..(11): flexible linker element added between T4-Foldon and CD95L-RBD in the T4-Foldon and mmCD95L-RBD fusion
GSSGSSGSSGS

SEQ ID NO: 18 - protein, 16 aa, Homo sapiens
  MISC_FEATURE (1)..(16): hexahistidine tag and a streptag-ll added C-terminally to T4-Foldon and mmCD95L-RBD fusion
HHHHHHSAWSHPQFEK

SEQ ID NO: 19 - protein, 8 aa, Homo sapiens
  MISC_FEATURE (1)..(8): flexible linker element used to join affinity tag (SEQ ID 18) to the T4-Foldon
SGPSSSSS

(19) United States
(12) Patent Application Publication
(10) Pub. No.: US 2011/0300113 A1
(43) Pub. Date: Dec. 8, 2011

(54) Death Receptor CD95 Controls Neurogenesis of Adult Neural Stem Cells in Vivo and in Vitro

(75) Inventors: Ana Martin-Villalba, Heidelberg (DE); Nina Corsini, Heidelberg (DE); Elisabeth Letellier, Heidelberg (DE); Susanne Kleber, Heidelberg (DE); Stefan Klussmann, Peine (DE)

(73) Assignee: DEUTSCHES KREBSFORSCHUNGSZENTRUM, Heidelberg (DE)

(21) Appl. No.: 12/442,017

(22) PCT Filed: Sep. 19, 2007
(86) PCT No.: PCT/EP2007/008167

§ 371 Date: Jun. 24, 2009

Related U.S. Application Data
(60) Provisional application No. 60/845,506, filed on Sep. 19, 2006.

(51) Int. Cl. A61K 35/12; C12N 5/10; A61K 31/7088; C07H 21/04; C07K 14/52; A61P 25/28; A61P 25/16; A61P 25/00; C12N 15/85; C07K 16/28

(52) U.S. Cl. 424/93.21; 435/320.1; 435/325; 514/44 R; 424/93.7; 530/389.6; 530/351; 536/23.5

(57) ABSTRACT

The present invention relates to the activation of the CD95L/CD95 system for inducing neuronal differentiation in vitro and in vivo and the use of CD95L compositions to effect differentiation.

### FIELD OF THE INVENTION

The present invention relates to the activation of the CD95L/CD95 system for inducing neuronal differentiation in vitro and in vivo and to methods and compositions to effect differentiation.

### BACKGROUND OF THE INVENTION

Neurogenesis is a multi-step process involving many different transcription factors and genes. One of the central questions in stem cell research remains understanding the gene programs that mediate maintenance and differentiation of neural stem cells, especially when biological tissue is injured or damaged. Typically, injury to the central nervous system (CNS) stimulates the production of neural stem cells (NSCs). These new cells migrate into the damaged area to replace injured cells. Most of these cells, however, die during the first weeks. Furthermore, few, if any, of the cells that do survive undergo neuronal differentiation and integration.

In the adult brain, neural stem cells exist in neurogenic regions; namely the subventricular zone (SVZ) of the lateral ventricles and the subgranular zone (SGZ) of the hippocampal dentate gyrus (DG)⁴. Differentiation of these cells into the different neural progenitors is regulated by the specialized microenvironment in which these cells reside, the neurogenic “niche”⁵. In the non-neurogenic adult cortex, targeted apoptotic degeneration of cortical neurons led to formation of new cortical neurons extending axons to the thalamus⁶.

Similarly, pathological conditions such as stroke or spinal cord injury, increase the production of NSCs, which ultimately replace only a small fraction of the mature injured neurons (roughly about 0.2%) due to their high mortality and poor neuronal differentiation. Taken together, conditions where only apoptosis takes place provide a neurogenic environment for NSCs. In such conditions neurotrophic factors and also pro-apoptotic molecules are upregulated⁸⁻¹¹.

One of these pro-apoptotic molecules is CD95L. The CD95 (APO-1 or Fas)/CD95L System has been extensively studied in the immune system as an inducer of apoptosis¹². Ligation of CD95 by trimerized CD95L leads to the subsequent recruitment of the adapter protein Fas-associated death domain (FADD, MORT1) and caspase-8 to the CD95 receptor. This results in the formation of a death-inducing signaling complex (DISC). Procaspase-8 at the DISC is activated through self-cleavage and commits the cell to apoptosis by activation of downstream effector caspases¹².

In immature neurons, CD95L promotes dendritic branching¹⁵. Mice deficient in CD95 or CD95L (lpr and gld mice, respectively) exhibit a decreased dendritic arborisation in hippocampal neurons. Desbarats et al. observed that stimulation of CD95 results in outgrowth of dorsal root ganglion cells in vitro¹⁶. The authors further demonstrated that lpr and gld mice were highly sensitive to neuronal degeneration in a model of Parkinson-like disease¹⁷.

CD95L expression is induced upon injury to the brain, such via a stroke^{9,28}. In instances of stroke, there typical is proliferation of NSCs that migrate and integrate into the region of the brain that has been injured. However, nearly all newborn neurons die after 5 weeks¹.

By contrast, inducing targeted apoptosis in selected neurons located in non-neurogenic regions results in proliferation, migration and integration of adult neural stem cells⁶. Consistently, when E17 neurons are transplanted into the apoptotic cortex they integrate and differentiate into functional mature neurons, but this is not the case if they are transplanted into the necrotic cortex²⁹.

Magavi et al. have shown that selective apoptotic degeneration of cortical neurons induces neuronal differentiation of transplanted and endogenous neural progenitors^{6,29}. A possible mechanism, they say, is that apoptosis induces expression of factors that control neuronal differentiation. Candidate molecules discussed are neurotrophins, e.g. BDNF, NT-3 and NT-4/-5¹¹. Another mechanism suggested is that apoptosis releases a repressed program of neural plasticity that permits and/or instructs early neocortical differentiation.

Optimizing the survival and differentiation of neural stem cells is one of the major goals in stem cell biology today. The present invention achieves this goal by uncovering further insights into the mechanisms controlling neurogenesis.

### SUMMARY OF THE INVENTION

An aspect of the present invention is the use of a composition comprising an activator of the CD95-Ligand (CD95L)/CD95-Receptor (CD95) system for the manufacture of a medicament for the treatment of a neuronal injury.

In one embodiment, the activator may be CD95L. In another embodiment, the activator may be an activating antibody directed against CD95.

The neuronal injury may be a neurodegenerative disease such as Parkinson's disease, Alzheimer's disease, or stroke or spinal cord injury. The treatment is preferably in a sub-acute stage of the injury. The treatment may induce neuronal differentiation in vitro or in vivo. Further, the treatment may comprise (i) an administration of the activator to neural stem cells, preferably to adult neural stem cells, and more preferably to adult human neural stem cells and (ii) introduction or migration of the treated stem cells to the site of neuronal injury.

Another aspect of the invention is the use of a composition comprising a polynucleotide encoding CD95L for the manufacture of a medicament for the treatment of a neuronal injury.

In one embodiment, the treatment comprises (i) an administration of the polynucleotide to neuronal stem cells and (ii) introduction or migration of the treated stem cells to the site of neuronal injury.

Another aspect of the present invention is a method for treating injured neuronal tissue, comprising: (i) treating stem cells with a CD95/CD95L activator and (ii) introducing those treated stem cells to the site of neuronal injury, wherein the treated stem cells differentiate into new neuronal cells at the site of injury.

In one embodiment, the neuronal tissue is in a mammal. In another embodiment, the mammal is a human patient who has a neuronal disorder, e.g. Parkinson's Disease, or has suffered from a neuronal injury, stroke or spinal cord injury.

In another embodiment, the injured neuronal tissue is in the brain of the mammal or elsewhere in the mammal's central nervous system.

In another aspect of the invention is a method for treating injured neuronal tissue, comprising: (i) engineering a stem cell to express a polynucleotide encoding CD95L, or a derivative of such a polynucleotide, and (ii) introducing that engineered stem cell into a subject, wherein the stem cell migrates to the site of injury and the polynucleotide is operably linked to regulatory elements that are preferably only active in the tissue that has been injured.

In another aspect of the invention is a method for treating injured neuronal tissue, comprising introducing a viral vector into a subject, wherein the vector comprises a polynucleotide that encodes CD95L, or a derivative of such a polynucleotide, and is operably linked to regulatory elements that are preferably only active in the tissue that has been injured. In one embodiment, the vector is an adenovirus associated vector or lentivirus vector.

In another aspect is a vector comprising a polynucleotide that encodes a CD95L protein, or a derivative of such a polynucleotide, wherein the polynucleotide is operably linked to (i) a promoter that is preferably active in neuronal tissue only and (ii) a terminator functional in neuronal tissue. In one embodiment, the CD95L protein comprises the sequence depicted in SEQ ID NO: 3. In another embodiment, the protein is encoded by the mRNA sequence of SEQ ID NO: 1 or the gene transcript sequence depicted in SEQ ID NO: 2.

In another aspect the invention refers to a stem cell, preferably to an adult stem cell, more preferably to a human adult stem cell, which comprises in its genome a non-naturally occurring polynucleotide that comprises a polynucleotide that encodes a CD95L protein, or a derivative of such a polynucleotide, wherein the polynucleotide is operably linked to (i) a promoter that is preferably active in neuronal tissue only and (ii) a terminator functional in neuronal tissue.

Both the foregoing summary and the following brief description of the drawings and the detailed description are exemplary and explanatory and are intended to provide further explanation of the invention as claimed. Other objects, advantages, and novel features will be readily apparent to those skilled in the art from the following detailed description of the invention.

### BRIEF DESCRIPTION OF THE DRAWINGS

FIG. 1: CD95 stimulation induces neurogenesis in mouse and human neural progenitor cells. (a) Treatment scheme for mouse neural progenitor cells. (b) CD95 stimulation via CD95L-LZ or αCD95AB generated cells positive for the neuronal marker β-III-tubulin in SVZ NSCs (light grey: isotype, dark grey: cells stimulated with control isotype). (c) CD95 stimulation as well as RA treatment increased the amount of β-III-tubulin positive hippocampal NSCs (light grey (lg): unstained cells, dark grey (dg): cells stimulated with control supernatant). (d) Treament scheme for human neural progenitor cells. (e) Treatment of human neural progenitors with CD95L-LZ or an agonistic antibody to CD95 (αApo1) led to increased generation of β-III-tubulin positive cells after growth factor removal. The graphs show the quantification of the increase in β-III-tubulin positive cells in human neural progenitor cells 4 days and 7 days after growth factor removal. Data are expressed as mean±S. D. of 5 display windows (student 's t-test, *p<0,05; **p<0,001).

FIG. 2: Characterisation of CD95L-induced neurogenesis. (a-f) Cells were treated as shown in FIG. 1a. Gene expression of mouse adult SVZ NSCs treated for 48 h with αCD95AB was analyzed by quantitative real-time PCR. Control cells: black bars; CD95-stimulated cells: grey bars.

FIG. 3: CD95L sequences—(a) mRNA (SEQ ID NO: 1), (b) DNA (SEQ ID NO: 2), and (c) protein (SEQ ID NO: 3) sequences for CD95L. The underlined portions of the sequence represents alternating exon structure; the enboldened glycine residues in Figure (c) represents residue overlap splicesite.

FIG. 4: Construction, development and properties of CD95L-T4. (a) Localisation of the N- and C-terminal amino acids within the TRAIL-RBD, forming an antiparallel β-strand.:Enlargement of the TRAIL-RBD-structure (N-terminus: green; C-terminus: red). (b) Position of the N- and C-terminus within the structure of the TRAIL-RBD. Upper row: side view of the central axis of the TRAIL-RBD. Lower row: top view of the central axis of the TRAIL-RBD. Left to right: mono-, di- and trimer. (c) mmCD95L-T4 amino acid sequence. The signal peptide is underlined (Met1-Gly20). The P141L-point mutation introduced into the murine CD95L-RBD to allow processing at E140 is marked by an asterix. The N-terminal (green arrow) and C-terminal amino acids (red arrow) of the mmCD95L-RBD are proposed to form an antiparallel β-strand. The T4-Foldon sequence is printed in blue (italics). (d) Affinity purification of mmCD95L-T4: mmCD95L-T4 purified by Streptactin affinity purification from transiently transfected Hek293T cells analysed by SDS-PAGE and silver staining. The position of mmCD95L-T4 is indicated. mmCD95L-T4 shows an apparent molecular weight of approximately 30 kDa. (e) Purification of trimeric CD95L-T4 by size exclusion chromatography (SEC): Fractions E2-E4 of affinity purified mmCD95L-T4 were separated by SEC using a Superdex 200 column. The graph shows the elution profile (OD 280 nm) of Streptactin-purified mmCD95L-T4. The main peak at 13.88 ml indicates the retention volume of trimeric mmCD95L-T4. Fraction A1-B1 collected from SEC (see B) were analysed by SDS-PAGE and silver staining. (f) Fractions A11 to A15 (B) containing putative trimeric mmCD95L-T4 were pooled, concentrated and subsequently analysed by SEC and SDS-PAGE silver staining. The analysis shows trimeric mmCD95L-T4 with a retention volume of 13.74 ml and indicates no significant aggregation of the protein. (g) Model of TRAIL-T4-DR5 complex. Upper Row: side view of TRAIL-DR5 co-complex with the T4-Foldon (light blue) positioned above the TRAIL-RBD (dark blue). The DR5 chains are coloured in dark red. The N-terminal amino acids of the TRAIL-RBD and the T4-Foldon are coloured in green; the C-terminal amino acids are coloured in red. Upper row: side view of the central axis of the TRAIL-RBD. Lower row: top view of the central axis of the TRAIL-RBD. Left: ribbon model. Right: surface model. (h) mmCD95L-T4 induces apoptosis in Jurkat cells in a dose dependent manner. (i) mmCD95L-T4 induces apoptosis in P815 cells in a dose dependent manner.

### DETAILED DESCRIPTION

The present invention relates to the discovery that the ligand for CD95 (APO-1/Fas), namely, CD95L, induces neuronal differentiation in vitro and in vivo and use of CD95L compositions to effect differentiation. The present invention demonstrates that activation of CD95 can promote neurogenesis in neural stem cells (NSCs) in areas of neuronal damage or injury. It is known that the central nervous system, when it is injured, produces various chemoattractants that attract stem cells to the damaged location. Those stem cells then differentiate, in part due to the CD95/CD95L interactions, into neural stem cells and, subsequently, neurons. See also Zuliani et al., Cell Death and Differentiation, (2006) 13, 31-40 (published online 8 Jul. 2005), which is incorporated herein by reference.

Indeed, it is shown herein that adult neural stem cells express CD95 on their surface but those cells do not undergo apoptosis after being treated with CD95L. Instead, it was found that stimulation of CD95 in murine and human NSCs induces neuronal differentiation. In mice subjected to global ischemia, GFP-labelled NSCs from mice deficient in CD95 (lpr) or wild type mice were injected into the striatum. In these mice, isolated neurons in the CA1 region of the hippocampus die upon CD95L³. Neuronal differentiation and integration within the ischemic CA1 region was only observed in case of wt NSCs. Lpr NSCs were detected in a neurogenic region, the dentate gyrus, but not in the ischemic CA1 region.

Thus, according to the present invention, an activator of the CD95L/CD95 system or a polynucleotide encoding CD95L can be used for the treatment of a neuronal injury. In a preferred embodiment the activator is CD95L, particularly human CD95L encoded by the nucleic acid sequence shown in SEQ ID NO: 2 or a nucleic acid sequence hybridizing under stringent conditions, e.g. after washing with 1×SSC, 0.1% SDS buffer at 55° C., preferably at 60° C. and more preferably at 65° C., or a fragment or derivative thereof. Preferred are also fusion proteins comprising at least one CD95L domain as described above and a heterologous polypeptide domain, preferably to an immunoglobulin Fc domain, such as a human immunoglobulin Fc domain, e.g. human IgG1 Fc. Further preferred are fusion proteins comprising a plurality, e.g. three CD95L domains, or fusion proteins comprising a CD95L domain and a multimerization domain, e.g. a trimerization domain, such as the Foldon-domain from bacteriophage T4.

In a further preferred embodiment, the activator is an activating antibody directed against the CD95 receptor. The activating antibody may bind to the CD95 receptor and thereby induce receptor activation. The antibody may be a monoclonal, chimeric, humanised or human antibody or an antigen-binding fragment or derivative thereof, e.g. a single-chain antibody or antibody fragment.

The active agent may be used for the treatment of neuronal injuries, e.g. neuronal diseases, particularly neurodegenerative diseases, or injuries of CNS, such as brain or spinal cord. The treatment is preferably carried out in a sub-acute stage of the disease or injury, particularly in a stage where CD95 expression is substantially absent in the injured tissue.

For instance, CD95L or an activatory antibody against CD95 could be used to create neurons in vitro from embryonic stem cells, which then can be introduced to the damaged tissue site. Or, stem cells may be engineered so that they overexpress a polynucleotide, which encodes the CD95 ligand, and then those cells could be injected into the patient. Once injected, the cells would migrate to the site of injury and express CD95L, thereby inducing in vivo neurogenesis. In a similar vein, a patient may be injected with a viral vector, such as an adenovirus or lentivirus vector, that has been engineered to express a CD95L-encoding polynucleotide. In such a construct, the polynucleotide would preferably be operably linked to regulatory elements that are active only in the tissue within which is the site of injury. Thus, expression of CD95L would not occur outside of the injured tissue. Hence, neurogenesis would occur at the site of injury.

At the time where CD95L has its highest expression—the acute injury phase—apoptosis is in most cases associated with massive induction of necrosis, inflammation, and production of reactive oxygen species. In this situation, the neurogenic program induced by CD95L might be altered or even absent. Death of neural stem cells following ischemia could therefore not be an active mechanism but simply a consequence of the absence of neurogenic cues.

The data described below suggest, that apart from being a useful tool for inducing neuronal differentiation of NSCs in vitro, CD95L can be used in vivo to endogenously repair the injured CNS.

### DEFINITIONS

The present invention is described herein using several definitions, as set forth below and throughout the application.

As used herein, “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

As used herein, the phrase “therapeutically effective amount” shall mean that drug dosage that provides the specific pharmacological response for which the drug is administered in a significant number of subjects in need of such treatment.

As used herein “patient” refers to a subject, preferably a human, who is in need of treatment.

“Recombinant proteins or polypeptides” refer to proteins or polypeptides produced by recombinant DNA techniques, i.e., produced from cells, microbial or mammalian, transformed by an exogenous recombinant DNA expression construct encoding the desired protein or polypeptide. Proteins or polypeptides expressed in most bacterial cultures will typically be free of glycan. Proteins or polypeptides expressed in yeast may have a glycosylation pattern different from that expressed in mammalian cells.

“Native” or “naturally occurring” proteins or polypeptides refer to proteins or polypeptides recovered from a source occurring in nature. The term “native” or “naturally occurring” includes native or naturally occurring polypeptides, and fragments thereof, and would include post-translational modifications of such polypeptides and fragments thereof, including, but not limited to, acetylation, carboxylation, glycosylation, phosphorylation, lipidation, acylation, and cleavage.

A DNA or polynucleotide “coding sequence” is a DNA or polynucleotide sequence that is transcribed into mRNA and translated into a polypeptide in a host cell when placed under the control of appropriate regulatory sequences. The boundaries of the coding sequence are the start codon at the 5′ N-terminus and the translation stop codon at the 3′ C-terminus. A coding sequence can include prokaryotic sequences, cDNA from eukaryotic mRNA, genomic DNA sequences from eukaryotic DNA, and synthetic DNA sequences. A transcription termination sequence will usually be located 3′ to the coding sequence.

“DNA or polynucleotide sequence” is a heteropolymer of deoxyribonucleotides (bases adenine, guanine, thymine, cytosine). DNA or polynucleotide sequences encoding the proteins or polypeptides of this invention can be assembled from synthetic cDNA-derived DNA fragments and short oligonucleotide linkers to provide a synthetic gene that is capable of being expressed in a recombinant DNA expression vector. In discussing the structure of particular double-stranded DNA molecules, sequences may be described herein according to the normal convention of providing only the sequence in the 5′ to 3′ direction along the non-transcribed strand of cDNA.

“Recombinant expression vector or plasmid” is a replicable DNA vector or plasmid construct used either to amplify or to express DNA encoding the proteins or polypeptides of the present invention. An expression vector or plasmid contains DNA control sequences and a coding sequence. DNA control sequences include promoter sequences, ribosome binding sites, polyadenylation signals, transcription termination sequences, upstream regulatory domains, and enhancers. Recombinant expression systems as defined herein will express the proteins or polypeptides of the invention upon induction of the regulatory elements.

“Transformed host cells” refer to cells that have been transformed and transfected with exogenous DNA. Exogenous DNA may or may not be integrated (i.e., covalently linked) to chromosomal DNA making up the genome of the host cell. In prokaryotes and yeast, for example, the exogenous DNA may be maintained on an episomal element, such as a plasmid, or stably integrated into chromosomal DNA. With respect to eukaryotic cells, a stably transformed cell is one which is the exogenous DNA has become integrated into the chromosome. This stability is demonstrated by the ability of the eukaryotic cell lines or clones to produce via replication a population of daughter cells containing the exogenous DNA. The terms “analog”, “fragment”, “derivative”, and “variant”, when referring to the polypeptides of this invention mean analogs, fragments, derivatives, and variants of such polypeptides that retain substantially similar functional activity or substantially the same biological function or activity as the polypeptides of the invention, as described herein.

An “analog” includes a pro-polypeptide that includes within it, the amino acid sequence of a polypeptide according to the invention. The polypeptide of the invention can be cleaved from the additional amino acids that complete the pro-polypeptide molecule by natural, in vivo processes, or by procedures well known in the art, such as by enzymatic or chemical cleavage.

A “fragment” is a portion of a polypeptide of the present invention that retains substantially similar functional activity or substantially the same biological function or activity as a polypeptide of the invention.

A “derivative” includes a polypeptide of the invention that substantially preserves the functions disclosed herein and include additional structure and attendant function, e.g., PEGylated polypeptides, which have greater half-life.

A “variant” includes polypeptides having an amino acid sequence sufficiently similar to the amino acid sequence of the original improved polypeptides of this invention. The term “sufficiently similar” means a first amino acid sequence that contains a sufficient or minimum number of identical or equivalent amino acid residues relative to a second amino acid sequence such that the first and second amino acid sequences have a common structural domain and/or common functional activity. For example, amino acid sequences that contain a common structural domain that is at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical are defined herein as sufficiently similar. Variants include variants of polypeptides encoded by a polynucleotide that hybridizes to a polynucleotide of this invention, or a complement thereof, under stringent conditions. Such variants generally retain the functional activity of the polypeptides of this invention. Variants include polypeptides that differ in amino acid sequence due to mutagenesis.

“Substantially similar functional activity” and “substantially the same biological function or activity” each means that the degree of biological activity is within about 50% to about 100% or more, preferably within about 80% to about 100% or more, and more preferably within about 90% to about 100% or more, of that biological activity demonstrated by the polypeptide to which it is being compared when the biological activity of each polypeptide is determined by the same procedure or assay.

“Similarity” between two polypeptides is determined by comparing the amino acid sequence of one polypeptide to the sequence of a second polypeptide. An amino acid of one polypeptide is similar to the corresponding amino acid of a second polypeptide if it is identical or a conservative amino acid substitution. Conservative substitutions include those described in Dayhoff, M. O., ed., The Atlas of Protein Sequence and Structure 5, National Biomedical Research Foundation, Washington, D.C. (1978), and in Argos, P. (1989) EMBO J. 8:779-785. For example, amino acids belonging to one of the following groups represent conservative changes or substitutions:

### Ala, Pro, Gly, Gln, Asn, Ser, Thr:

### Cys, Ser, Tyr, Thr;

### Val, Ile, Leu, Met, Ala, Phe;

### Lys, Arg, H is;

### Phe, Tyr, Trp, His; and

### Asp, Glu.

“Mammal” includes humans and domesticated animals, such as cats, dogs, swine, cattle, sheep, goats, horses, rabbits, and the like.

All other technical terms used herein have the same meaning as is commonly used by those skilled in the art to which the present invention belongs.

It is to be understood that the examples are for illustrative purposes only, and should not be interpreted as restricting the spirit and scope of the invention, as defined by the scope of the claims that follow. All references identified herein, including U.S. patents, are hereby expressly incorporated by reference.

### EXAMPLES

### Material and Methods

Global ischemia. Briefly, 10 week old female C57BL6 mice were anesthetized and common carotoid arteries were exposed. Both carotoid arteries were occluded using micro clamps (Fine Science Tools). After 10 min clamps were removed and the wound was closed. 48 hours after global ischemia induction, mice were anesthetised. NSCs were infected with the lentiviral vector pEIGW at a MOI of 5. All lentiviruses were propagated using previously described methods. Expression of eGFP was confirmed in infected cells by flow cytometric analysis. NSCs were trysinised, washed in PBS and resuspended at a concentration of 250000 cells per μl medium. 2 μl of the cell suspension was injected into the striatum using a Flexi-fill syringe (World Precision Instruments) and a Micro4 syringe pump controller (World Precision Instruments) at a rate of 200 nl/min. For histology, FACS analysis or Real-time PCR 3 mice per group were used, for behavioural tests 13 mice per group were used.

Real-time Quantitative PCR. 24 h cells after plating cells were stimulated with 1 activation unit of CD95L-T4. After 48 h RNA was isolated using the RNeasy Mini Kit (Qiagen). Real-time quantitative PCR was carried out using Sybr Green core kits (Eurogentec) and Uracil-N-glycosylase (Eurogentec). For determining CD95L induction after global ischemia, the hippocampi were dissected out and stored in RNAlater reagent (Ambion). RNA was extracted using the RNeasy Mini Kit (Qiagen). Real-time quantitative PCR was carried out using Sybr Green core kits (Eurogentec) and Uracil-N-glycosylase (Eurogentec). Primers used for quantitative real-time PCR were designed using Primer 3 software (http://fokker.wi.mit.edu/cgi-bin/primer3/primer3_www.cgi).

| | | | | |
|---|---|---|---|---|
| Cyclin D2 | forward | 5′-tgatggtttgggaagtttga-3′ | reverse | 5′-gtgcgtgagctctggataaa-3′ |
| Nestin | forward | 5′-cttgcagacacctggaagaa-3′ | reverse | 5′-cctctggtatcccaaggaaa-3′ |
| Jagged | forward | 5′-cagaatgggaactgttgtgg-3′ | reverse | 5′-ctccttgaggcacactttga-3′ |
| BDNF | forward | 5′-tggttactctcctgggttcc-3′ | reverse | 5′-ttcactccctgagtcacagc-3′ |
| Neurogenin | forward | 5′-gagccggctgacaatacaat-3′ | reverse | 5′-ctcaggttcttcctggagca-3′ |
| Gapdh | forward | 5′-attcaacggcacagtcaagg-3′ | reverse | 5′-tggatgcagggatgatgttc-3′ |

Lentiviral infections. SVZ NSCs were infected with the lentiviral vector pEIGW and pEIGW-GSK3βS9A at a multiplicity of infection (MOI) of 5. The plasmids were constructed by replacing the eGFP sequence between the EF1α promotor and the WPRE element in pWPTS-eGFP with the IRES-eGFP cassette form pIRES2-eGFP (Clontech, Germany). The recombinant lentiviral vector pEIGW-GSK3βS9A was constructed using pcDNA3 HA-GSK3βS9A. This vector encodes a constitutively active GSK3β mutant containing a serine-to-alanine substitution at residue 9 (GSK3βS9A). Expression of all transgenes was confirmed in infected cells by FACS analysis of GFP expression. The percentage of infected cells was 70-90%.

Immunostainings. Immunohistochemistry and Immunocytochemistry were performed as previously described. Primary antibodies used were anti GFP (chicken) (Ayes Labs) 1:1000, anti NeuN (mouse), (Chemicon) 1:200, anti β-III-tubulin (mouse) (Chemicon 1:200), anti GFAP (mouse) (Chemicon 1:200), active-β-Catenin (mouse) (P-Ser37 or P-Thr41; 1:800) (Upstate), phosphorylated GSK3β (P-Ser9-GSK3β, 1:1000). The mouse Pax6 antibody developed by A. Kawakami was obtained from the Developmental Studies Hybridoma Bank developed under auspices of the NICHD and maintained by The University of Iowa, Department of Biological Sciences, Iowa City, Iowa 52242. Secondary antibodies used were goat anti-rabbit alexa 488 (Molecular Probes) 1:500, goat anti-mouse rhodamine X (Dianova) 1:200, Dapi (Sigma) 1:3000, donkey anti-chicken FITC (Dianova) 1:200.

Western Blots. Phosphorylated GSK3β (P-Ser9-GSK3β, 1:1000), phosphorylated AKT (P-Ser473-KT, 1:1000), total AKT (T-AKT, 1:1000), Src family kinases (Src, 1:1000), cleaved caspase 3 (1:250), total β-Catenin (1:1000) and phosphorylated src (Tyr416) were purchased from Cell Signaling/New England Biolabs. pp60(c-Src) antibody was purchased from Upstate. Antibody against total GSK3β (T-GSK3β, 1:1000), were purchased from Santa Cruz. The PI3K inhibitor Ly 290059 (25 μM) and the Src family kinase inhibitor PP2 (20 μM) were purchased from Calbiochem. Cells were preincubated with inhibitors 30 minutes (LY 294002) and 1 hour (PP2) prior to treatment. Cells were lysed for further biochemical analysis. Protein extraction and immunoblotting was performed as previously described.

P13 Kinase activity assay. The PI3K activity assay was purchased from Echelon biosciences (K-1000). PI3K activity was measured according to the manufacturers instructions. For immunoprecipitations an antibody against p85 (2.5 μg+30 μl beads) and 50 μg protein were used.

Immunoprecipitation. 2×10⁷ cells were either treated with 1 activation unit (5-40 ng/ml depending on the batch of CD95L-T4) or 5000 ng/ml CD95L-T4 for 1 and 5 minutes (unless otherwise indicated) at 37° C. or left untreated, washed twice in PBS plus phosphatase inhibitors (NaF, NaN3, pNPP, NaPPi, β-Glycerolphosphate, 10 mM each and 2 mM orthovanadate), and subsequently lysed in buffer A (20 mM Tris/HCl, pH 7.5, 150 mM NaCl, 2 mM EDTA, 1 mM phenylmethylsulfonyl fluoride, protease inhibitor cocktail (Roche), 1% Triton X-100 (Serva, Heidelberg, Germany), 10% glycerol, and phosphatase inhibitors (NaF, NaN3, pNPP, NaPPi, 1′-Glycerolphosphate, 10 mM each and 2 mM orthovanadate) (stimulated condition) or lysed without treatment (unstimulated condition). Protein concentration was determined using BCA kit (Pierce). 1 mg of protein was immunoprecipitated overnight with either 5 μg anti-CD95 AB Jo2 or 5 μg anti-phospho tyrosine antibody (pTyr 4G10 (Upstate)) and 30 μl protein-A Sepharose. Beads were washed 5 times with 20 volumes of lysis buffer. The immunoprecipitates were mixed with 60 μl of Laemmli buffer and 30 μl were analyzed on either 15% or 7.5% SDS-PAGE. Subsequently, the gels were transferred to Hybond nitrocellulose membrane (Amersham Pharmacia Biotech, Freiburg, Germany), blocked with 2% BSA in PBS/Tween (PBS plus 0.05% Tween 20) for 1 hour, and incubated with the primary antibody in 2% BSA PBS/Tween at 4° C. overnight. Blots were developed with a chemoluminescence method following the manufacturer's protocol (PerkinElmer Life Sciences, Rodgan, Germany).

siRNA. SVZ NSCs were transfected with pp60-src ON-TARGETplus SMARTpool siRNA (100 nM) and non-targeting SMARTpool siRNA (100 nM) using Lipofectamine 2000 according to the manufacturers instructions. Briefly, cells were cultured in normal medium without antibiotics. Before transfection, cells were washed twice using Neurobasal A medium (Invitrogen). Transfection mix was prepared according to the manufacturers instructions. 6 hours after transfections, cells were fed with normal medium. 48 h later pp60 src knockdown was assessed by Western Blot. At the same time, cells were stimulated with CD95L-T4 and analysed after 48 h for β-III-tubulin positive cells.

Flow Cytometry. For staining of surface markers, cells were harvested using trypsin, washed in PBS containing 5% FCS and incubated with fluorochrome labelled antibodies or matching isotypes for 30 min on ice. Antibodies used were: anti-mouse CD95 PE (Becton Dickinson) 1:100, anti-mouse CD95L PE (Becton Dickinson) 1:50, isotype hamster PE (Caltag) 1:20, α-Apo1 (100 μg/ml), goat anti-mouse PE 1:33 (Dianova).

For staining of intracellular markers, cells were harvested, fixed with 4% PFA for 10 min on ice, washed and permeabilized using 0.1% Saponin (Sigma-Aldrich) in PBS with 10% FCS (SAP). Cells were incubated with primary antibody or isotype for 30 min on ice. Cells were washed three time with SAP and incubated with secondary antibody for 30 min on ice. Cells were washed again three times with SAP and analysed. Antibodies used were: anti β-III-tubulin (Chemicon) 1:100, anti GFAP (Chemicon) 1:100 and isotype mouse IgG1 (Sigma) 1:20. Secondary antibody was goat anti-mouse PE 1:100 (Dianova). To quantify DNA fragmentation, cells detached with trypsin were centrifuged, washed and fixed with 70% ethanol at −20° C. for 1 h. Fixed cells were resuspended in a hypotonic lysis buffer (0.1% sodium citrate and 0.1% Triton X-100) containing 50 mg/ml of propidium iodide, incubated at 4° C. overnight. After final washings, all samples were analysed using a FACS-Calibur (Becton Dickinson) and CellQuest Software (Becton Dickinson).

For determining CD95L induction after global ischemia, the hippocampus was dissected out, the tissue was trypsinized and homogenized using flame-polished pasteur pipettes. Cells were then incubated with anti-mouse CD95L PE (MFL3) (Becton Dickinson) 1:50 and isotype hamster PE (Caltag) 1:20 for 30 min on ice. After washing, samples were analysed using a FACS-Calibur (Becton Dickinson) and CellQuest Software (Becton Dickinson). For all FACS analysis done at least 100.000 events were counted.

Behavioural tests. All behavioural tests were performed as described before. The modified open-field test was performed 3 weeks after injection of NSCs mice after a 4 days period of habituation. Spontaneous alternation was tested 5 weeks after injection of NSCs. Spontaneous alternation was performed essentially like the T-maze with slight modifications. Only one session per day was performed, with one session consisting of four trials. In total two trials per mouse were performed. No food reward was given and mice were not deprived of food. The T-maze test assessing spatial working memory was done 3.5 weeks after injection of NSCs, followed by the Y-maze test (spatial memory).

Caspase Activity Assay. Apostat Intracellular Caspase Detection Caspase Activity Assay was purchased from R&D Systems. Assay was performed according to the manufacturers' instructions and analyzed using a FACSCalibur (Becton Dickinson) and CellQuest software (Becton Dickinson).

Statistical analysis. For samples in which n<5, statistical analyses were performed using the unpaired Student's t-test. For samples in which n>5, statistical analyses were performed using Kruskall-Wallis analysis.

Mouse adult neural stem cell culture. NSCs were isolated as previously described³⁰. Briefly, 6 week old female C57BL6 mice were sacrificied and tissue from hippocampus, SVZ and spinal cord was dissected out. The tissue was digested, washed and cultured in Neurobasal Medium (Invitrogen Karlsruhe, Germany) supplemented with B27 (Invitrogen), Penicillin-Streptomycin (Invitrogen), Glutamax (Invitrogen), Heparin (2 μg/ml) (Sigma) and the growth factors EGF (20 ng/ml) (Promocell) and bFGF (20 ng/ml) (Promocell). After 7 days in culture neurospheres were harvested by centrifugation, dissociated using trypsin (Invitrogen) and replated. Cells were passaged once to twice weekly. Cells used for experiments were passaged between 10 to 15 times. Cells were stimulated with 10 μM Retinoic Acid or 1 activation unit of CD95L-T4. One activation unit was defined as the amount of CD95L-T4 required to increase phosphorylation of GSK3β. Activation units were defined freshly for each batch of CD95L-T4 and varied from 5-40 ng/ml.

Human neural progenitor generation and culture. The human ES cell line H9.2 was cultivated on mitotically inactivated mouse embryonic fibroblasts (MEFs) in Knockout Dulbecco's Modified Eagle Medium (KO-DMEM, Gibco) containing 20% Knockout Serum Replacement, 1% non-essential amino acids, 1 mM I-glutamine, 0.1 mM β-mercaptoethanol (all from Gibco), and 4 ng/ml human basic fibroblast growth factor (FGF-2, Invitrogen). Cells were routinely passaged every four days by treatment with 100 units collagenase IV (Gibco) for one hour. For neural differentiation, 7 days-old embryoid bodies were transferred to polyornithine-coated tissue culture plates and propagated in DMEM:F12 (Invitrogen,) supplemented with 25 μg/ml insuline, 100 μg/ml transferrin, 30 nM sodium selenite (Sigma, Deisenhofen, Germany), 2.5 μg/ml fibronectin (R&D Systems, Wiesbaden, Germany) and 20 ng/ml FGF-2. Within 10 days, neural tube-like structures developed in the embyoid body outgrowth. These structures were mechanically isolated and propagated as free-floating neurospheres in N2 medium (DMEM:F12, 1×N2 supplement (Gibco), 2 μg/ml heparin) containing 10 ng/ml FGF-2 in petri dishes on a shaker for 2 weeks. Neural precursor cells were dissociated into single cells by trypsin digestion and plated on polyornithine/laminine-coated tissue culture plates in the presence of 10 ng/ml FGF-2 and 10 ng/ml EGF. Cells were treated as depicted with 1 μg/ml αApo1 or 0.5 U/ml CD95L-LZ¹⁵.

E15 primary neurons culture. Primary neurons were derived from E15 embryos and cultured as described¹⁵. In brief, the hippocampi of embryonic day 15 (E15) mice were dissected, trypsinized, and physically dissociated. Cells were washed in HBSS and 450.000 hippocampal or cortical cells were plated onto 3 cm tissue dishes containing Minimal Essential Medium (MEM) and 10% heat-inactivated horse serum. The cells were kept in 5% CO₂ at 36.5° C. After 4 hr, the medium was exchanged to medium containing MEM-N2 with B27 supplement (Gibco). After two days in culture, neurons were stimulated for 5 min with recombinant CD95 Ligand 200 ng/ml (Sigma).

Focal Ischemic animal model. In wild-type C57BL/6 mice, all matched for age (mean 100 days) and weight (mean 24 g), focal cerebral ischemia was induced by occlusion of the middle cerebral artery (MCA) as described previously²⁸. A surgical nylon thread was advanced from the lumen of the common carotid artery up to the anterior cerebral artery to block the origin of the MCA for 90 min. MCA blood flow was restored by withdrawing the nylon thread. Deep anesthesia was reached by Ketamin and Rompun (150 mg/kg body weight each). Animals were kept under anesthesia and rectal temperature was controlled at or near 37° C. with a heating lamp throughout both the surgical procedure and the MCA occlusion period up to the time the animals recovered from anesthesia. After a six-hour reperfusion period animals were deeply anesthesized and killed by decapitation. Thereafter different regions from the ischemic and non-ischemic (contralateral) brain hemisphere were homogenized for protein biochemistry

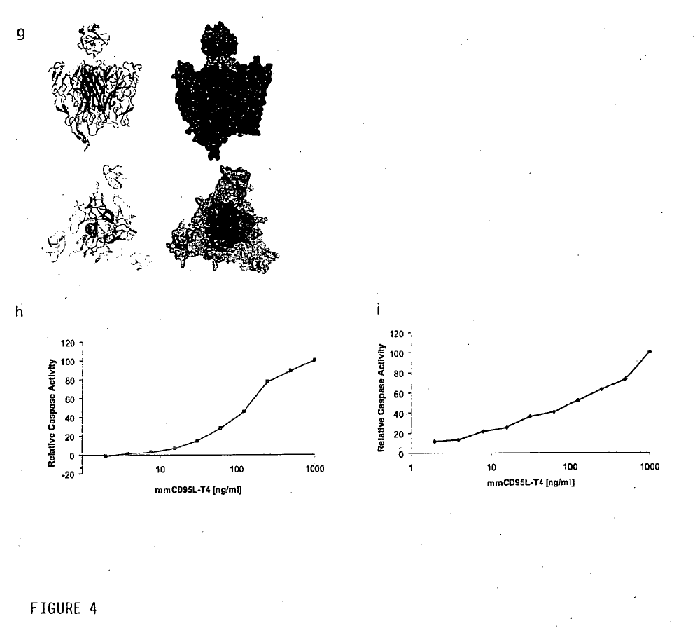

Genetic engineering of mus musculus CD95-Ligand-T4 (mmCD95L-T4). The TRAIL/DR5 complex as well as the TNF-α structure were used as models to develop expression strategies for the mus musculus CD95L-receptor binding domain (mmCD95L-RBD). Provided that the structure of trimeric mmCD95L-RBD is in principle similar to the TNF-α- or TRAIL-RBD-structures (PDB-entries: 1TNF and 1D0G/1DU3, respectively)³¹⁻³³, the following observations were taken into account: The N- and C-terminal amino acids of the RBD from TRAIL and TNF-α form an antiparallel β-strand. The terminal amino acids of this β-strand are located next to each other at the same site of the molecule close to the central axis of the TRAIL-RBD trimer (FIG. 4). This means, that for steric reasons, the use of N- and C-termini in the same molecule for the fusion of protein domains (e.g. for the addition of stabilisation motifs or tags) is mutually exclusive. The ideal stabilisation motif should be a small, well defined trimer located close to the central axis of the CD95L-trimer with its N- and C-terminus at opposite sites of the stabilisation motif, in order to minimize its risk of interference with the ligand/receptor interaction sites. An appropriate trimeric protein domain fulfilling these criteria is the T4-Foldon motif from the fibritin of the bacteriophage T4^{34,35}.

According to the above mentioned considerations the T4-Foldon was fused C-terminally to the mmCD95L-RBD (Glu140-Leu279 of mmCD95L). Between the CD95L-RBD and the T4-Foldon, a flexible linker element (GSSGSSGSSGS) was placed and a hexahistidine tag and a streptag-II (HHHHHHSAWSHPQFEK) was added C-terminally. This affinity tag was linked to the T4-Foldon by a flexible linker element (SGPSSSSS). To allow for secretory based expression, a signal peptide from human Igκ was fused to the N-terminus (Glu140). The proposed signal peptide cleavage site formed by the fusion of the Igκ leader to the mmCD95L-RBD is expected to release a final product with a N-terminal located Glutamine, corresponding to Glu140 of mmCD95L. To ensure correct processing, a single amino acid exchange close to the processing site was introduced, namely Pro141 was mutated to Leu (P140L). The amino acid sequence of the mmCD95L-T4-construct shown in FIG. 4 was backtranslated and its codon usage optimised for mammalian cell-based expression. Gene synthesis was done by ENTELECHON GmbH (Regensburg, Germany). The final expression cassette was subcloned into pcDNA4-HisMax-backbone, using unique Hind-III- and Not-I-sites of the plasmid. A schematic summary, including all features described above, is shown exemplarily for the TRAIL-T4-DR5-complex (FIG. 4).

Expression and Purification of mouse CD95-T4 Ligand (mmCD95L-T4). Hek 293T cells grown in DMEM+GlutaMAX (GibCo) supplemented with 10% FBS, 100 units/ml Penicillin and 100 μg/ml Streptomycin were transfected with a plasmid encoding mmCD95L-T4. Cell culture supernatant containing recombinant mmCD95L-T4 was harvested three days post transfection and clarified by centrifugation at 300 g followed by filtration through a 0.22 μm sterile filter. For affinity purification 1 ml Streptactin Sepharose (IBA GmbH, Gottingen, Germany) was packed to a column and equilibrated with 15 ml buffer W (100 mM Tris-HCl, 150 mM NaCl pH 8.0). The cell culture supernatant was applied to the column with a flow rate of 4 ml/min. Subsequently, the column was washed with buffer W and bound mmCD95L-T4 was eluted with buffer E (100 mM Tris-HCl, 150 mM NaCl, 2.5 mM Desthiobiotin, pH 8.0). The protein content of the eluate fractions was analysed by SDS-PAGE and silver staining. Fractions E2-E4 were subsequently concentrated by ultrafiltration and further purified by size exclusion chromatography (SEC).

SEC was performed on a Superdex 200 column (GE-Healthcare) equilibrated with phosphate buffered saline and the concentrated, streptactin purified mmCD95L-T4 (E2-E4) was loaded onto the SEC column at a flow rate of 0.5 ml/min. The elution of mmCD95L-T4 was monitored by absorbance at 280 nm. To purify defined trimeric mmCD95L-T4 the fractions A11 to A15 of the SEC were pooled, concentrated and a small amount of the pooled protein was again analysed by SEC.

The apparent molecular weight of the final purified mmCD95L-T4 was determined based on calibration of the Superdex 200 column with gel filtration standard proteins (Bio-Rad GmbH, München, Germany).

For experiments with NSCs an activation unit was defined. One activation unit (1 U) was the dose of CD95L-T4 required to phosphorylate AKT at 5 minutes. The activation unit varied between batches from 5-40 ng/ml.

### Example 1

Neural progenitor cells were first isolated from adult mouse SVZ, HC or spinal cord. Neural progenitor cells from all three regions expressed CD95 on the surface (data not shown). As already reported by others^{13,14}, these cells were resistant to CD95-mediated apoptosis (data not shown). It was then addressed whether an alternative non-apoptotic signalling downstream of CD95 might exist. Recently CD95 has emerged as a molecule involved in non-apoptotic signalling in the nervous system.

To study whether CD95L also has a non-apoptotic function in neural progenitors, it was determined if proliferation was changed upon CD95L-stimulation. Treatment of neural progenitor cells with CD95L or an anti-CD95 stimulating antibody did not lead to significant changes in the proliferation rate, when compared to untreated cells (data not shown).

### Example 2

Neural progenitor cells are characterized by their ability to self-renew and to differentiate into the various neural lineages. To determine if CD95L influences the latter, the expression of the neuronal marker β-III-tubulin and the glial marker GFAP was examined.

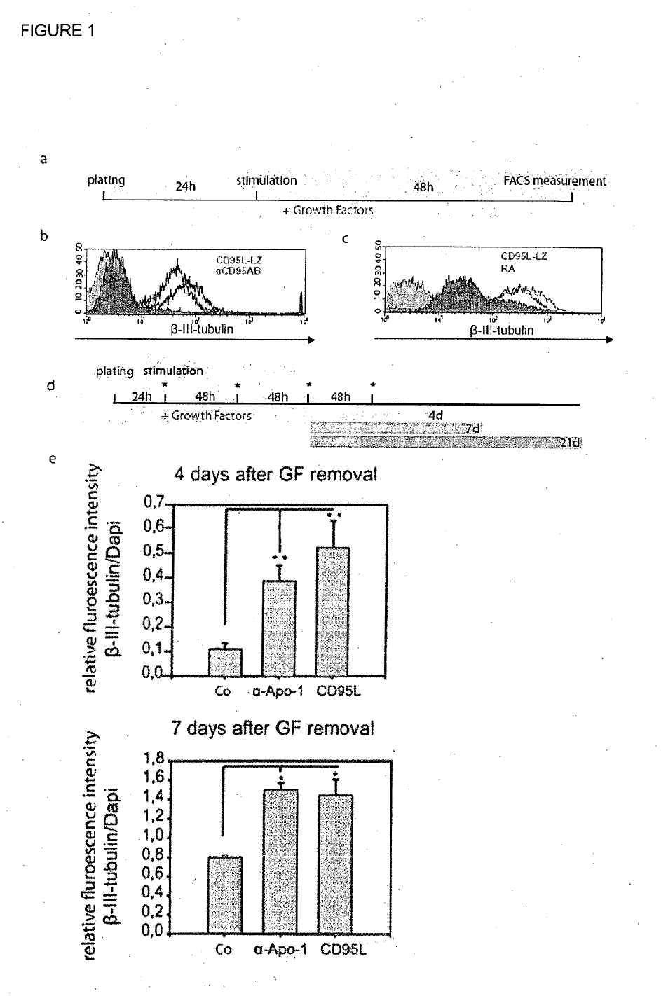

In neural SVZ progenitors triggering of CD95 in the presence of growth factors greatly increased β-III-tubulin expression (FIG. 1b). This effect was seen with both CD95L and an anti-CD95 stimulating antibody. Further, CD95L-induced neurogenesis could be blocked with a neutralizing antobody to CD95L (Nok1) (data not shown). Retinoic acid (RA) is a key regulator of adult neurogenesis in the DG and is commonly used to differentiate a number of different stem cells including adult neural stem cells into neurons¹⁸. Consistently, an increase in β-III-tubulin expression was observed upon treatment of neural progenitors isolated from the HC with retionic acid and CD95L (FIG. 1c).

To assess if CD95L is not only involved in induction of neuronal but also glial differentiation, the expression of GFAP was examined. CNTF was used as an inducer of astrocytic differentiation. CNTF has been shown to induce rapid and efficient differentiation of neural stem cells into GFAP-expressing astrocytic cells. Treatment of neural progenitor cells with CNTF led to an enhanced expression of GFAP. CD95L or RA treatment did not change GFAP expression (data not shown).

These results indicate that CD95L is not involved in induction of glial differentiation. Next, SVZ stem cells isolated from lpr mice were utilized. Lpr mice carry a natural mutation in the Tnfrsf6 (CD95) gene on chromosome 19 caused by the insertion of the early transposable element ETn²⁰. lpr mice therefore show reduced expression levels of CD95 on the surface of cells (<10%)(data not shown).

In these cells we observed increased expression of β-III-tubulin only with RA but not with an anti-CD95 AB, indicating that these cells per se have no differentiation defect (data not shown). Cells isolated from the brain of E11 embryos showed reduced levels of CD95 and did not show increased expression of β-III-tubulin levels (data not shown), suggesting that CD95L-mediated non-apoptotic signaling is not operative in E11 neural stem cells. In contrast, cells isolated from a later embryonic stage, namely E15, again showed increased β-III-tubulin expression upon CD95 stimulation (data not shown).

### Example 3

To address whether CD95L can induce neuronal differentiation also in human neuronal stem cells (NSCs), human neural progenitor cells derived from embryonic stem cells were utilized. Human neural progenitor cells expressed CD95 at similar levels compared to mouse NSCs (data not shown). Triggering of CD95 did not induce apoptosis in human neural progenitor cells (data not shown). These data confirm that human neural progenitor cells are also not sensitive to CD95L-induced apoptosis.

Thus, the lineage differentiation of this population was evaluated (FIG. 1d). Differentiation of neural progenitor cells commonly follows removal of growth factors that support self-renewal and proliferation. Consistently, four (4) days after growth factor withdrawal stem cells spontaneously gave rise to neurons as assessed by expression of β-III-tubulin. Treatment with both CD95L and an agonistic AB to CD95 (α-Apo1) resulted in a strikingly increased number of neurons (FIG. 1d). The increase in the relative fluorescence of the neuronal marker β-III-tubulin depicted in FIG. 1c was not due to an increase in branching of neurites, an effect known to be mediated by CD95L, but to an actual increase in neuronal numbers (data not shown). At 7 and 21 days after growth factor removal, we could also observe increased neuronal numbers (FIG. 1c). These findings indicate that CD95L induces neurogenesis also in human neural stem cells.

### Example 4

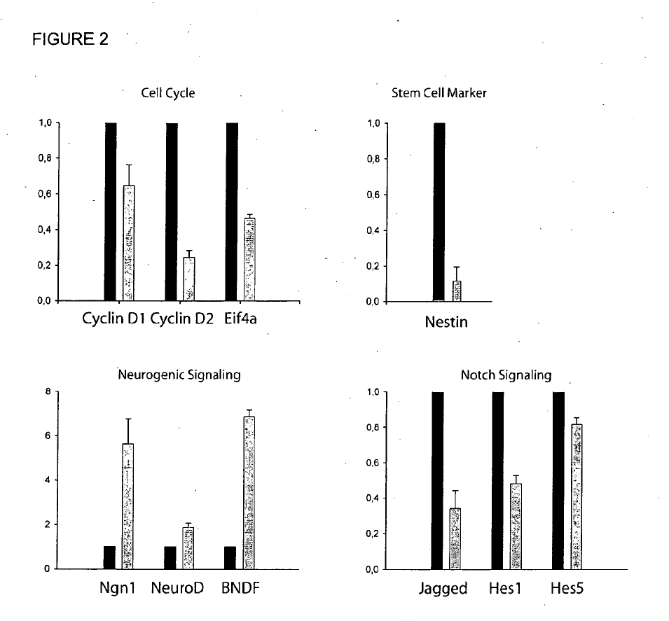

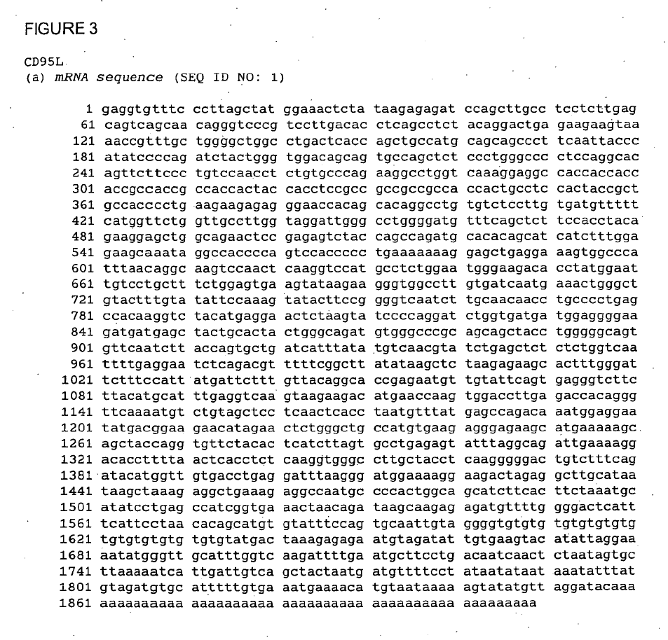

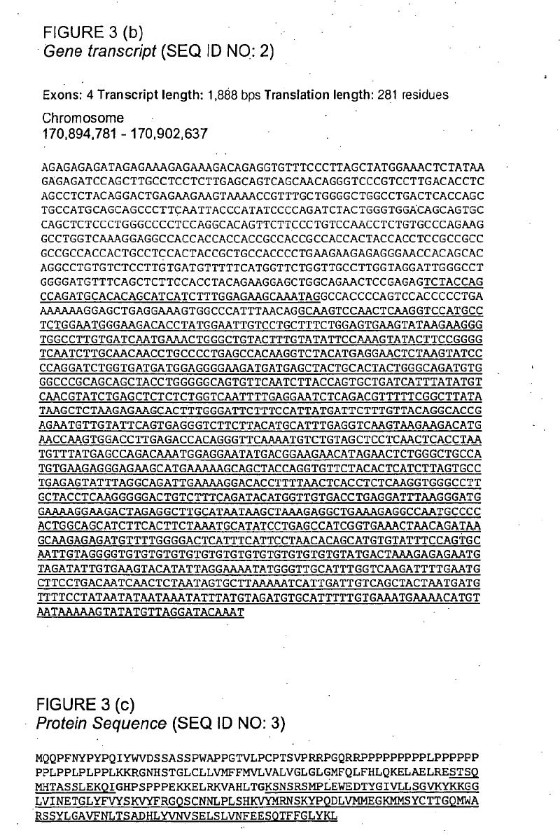

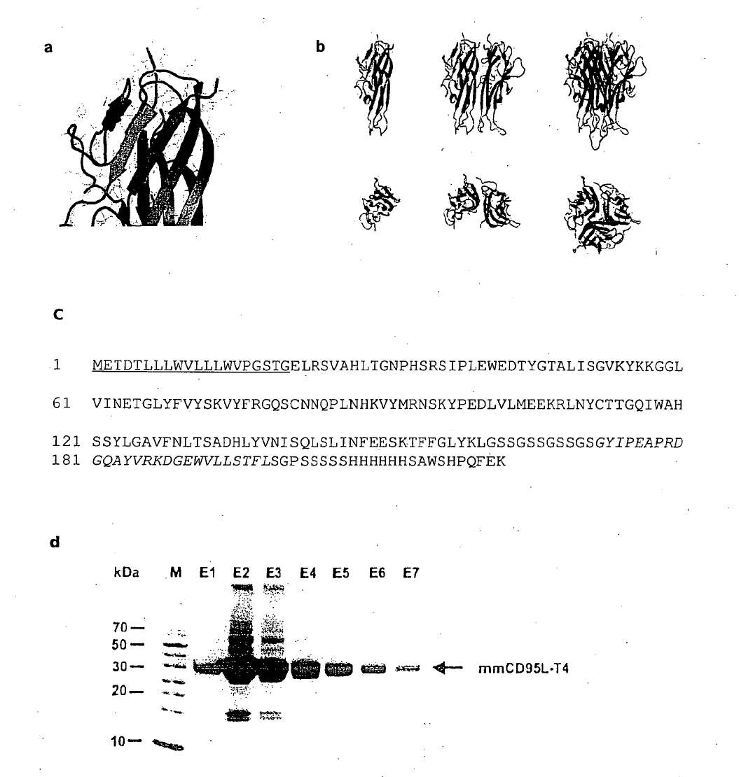

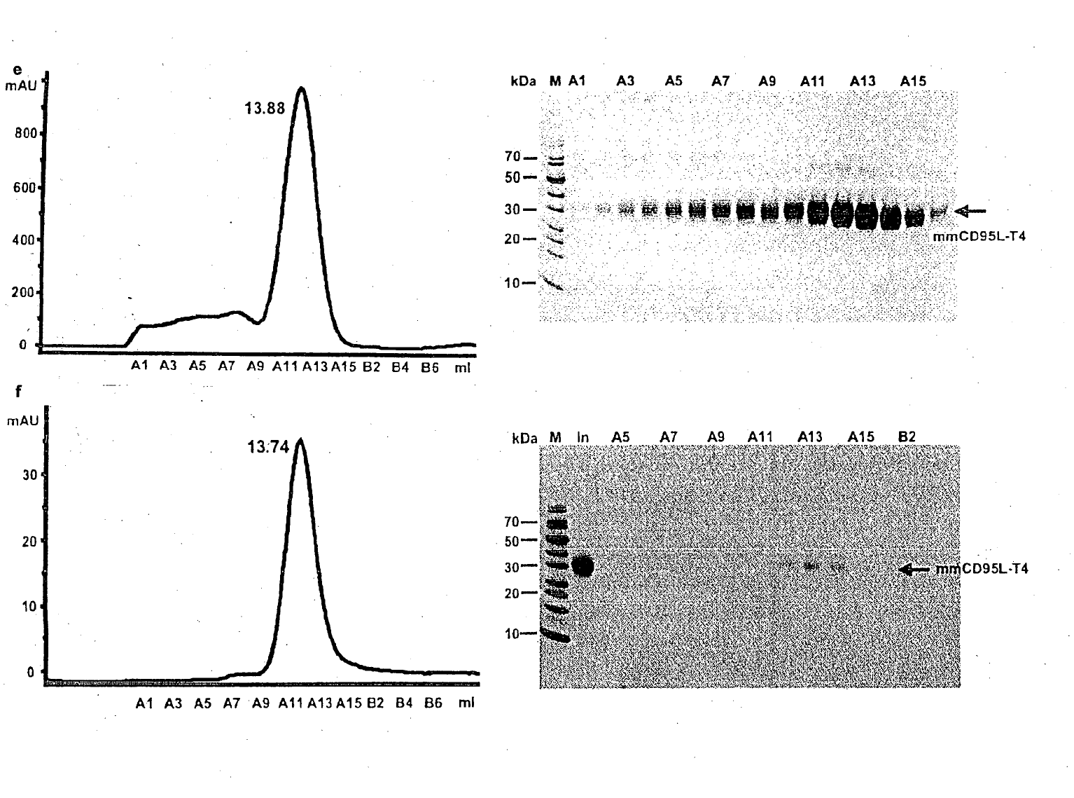

Neurogenesis is a multi-step process involving many different transcription factors and genes. One of the central questions in stem cell research remains understanding the gene programs that mediate maintenance and differentiation of neural stem cells. To further characterize the events leading to neuronal commitment by triggering of CD95, we determined mRNA levels of factors known to be involved in stem cell maintenance and differentiation. Inducers of neuronal and glial differentiation were used for comparative purposes. mRNA levels were analysed 48 h after stimulation. Cells treated with anti-CD95 antibody strongly decreased expression of Nestin and Cyclin D2 (FIG. 2). Nestin is a well-studied marker of neural stem cells²¹. Cyclins are key players in cell-cycle regulation²². The data demonstrate that CD95 stimulation results in loss of stem cell properties and promotes cell cycle exit, a prerequisite for differentiation, in neural progenitor cells. Accordingly, similar effects were seen with RA treatment. These data also suggest that CD95L does not promote the expression of selected neuronal markers (e.g. β-III-tubulin) in progenitor cells but actually induces the process of neuronal differentiation.

A marked downregulation of the Notch ligand Jagged1 in anti-CD95 AB treated cells was also observed. Notch signalling is known to inhibit neuronal differentiation and has an important role in neural stem cell expansion. Downregulation of Notch ligands might be a means for neural stem cells to escape Notch signalling and allow them to undergo the transition from stem cell expansion to differentiation. Jagged1 mRNA levels were also decreased upon stimulation with CNTF but not RA. These results indicate that CD95 stimulation in neural stem cells leads to downregulation of mechanisms responsible for maintenance and expansion of stem cells.

Treatment of neural stem cells with anti-CD95 antibody further resulted in decreased expression of Doublecortin like kinase1 (DCLK) and marked upregulation of Neurogenin (Ngn1)) (FIG. 2 and data not shown). In neural progenitor cells, the bHLH transcription factor Ngn1 simultaneously promotes neurogenesis and inhibits gliogenesis. DCLK is expressed in regions of active neurogenesis in the neocortex and in vivo loss of DCLK function confers neuronal differentiation to neural progenitor cells in the SVZ. The data demonstrrate that CD95 stimulation activates proneural gene expression programs. Consistently, treatment with RA had similar effects, although the extent of Ngn1 expression was much higher in CD95L-compared to RA-treated cells, indicating that CD95L is a more potent inducer of neurogenesis. In contrast, CNTF treatment had no effect on DCLK expression and resulted in an almost complete loss of Ngn1 mRNA, a further hint that CD95L promotes neuronal but not glial differentiation.

After treating neural progenitor cells with anti-CD95 antibody, a strong induction of brain-derived nerve factor (BDNF) was observed (FIG. 2). BDNF expression was also induced upon treatment with CNTF, but not with RA. BDNF is a well-studied neurotrophin that promotes cell survival and differentiation of neuronal cells. While it has little effect on adult neural stem cell cultures on its own, it regulates neurogenesis in collaboration with RA. RA promotes acquisition of a neuronal fate and sensitizes the cells toward BDNF by induction of Trk receptors. Subsequently, BDNF promotes maturation of immature neurons. BDNF induction by CD95 stimulation could account for a further differentiation of neural progenitor cells into more mature neurons as seen in our experiments. It is hypothesized that CD95 stimulation has a dual role: it could prime neural progenitor cells in a manner similar to RA and via BDNF lead to further maturation of newly generated neurons. In the tested system, treatment of neural stem cells with exogenous BDNF alone did not increase β-III-tubulin expression (data not shown).

### Example 5

Another key regulator of neurogenesis is the transcription factor Pax6. In cultured neurospheres, Pax6 promotes progression of neural progenitor cells towards neuronal lineage. Expression of Pax6 in vivo is first detected when precursor cells from the subependymal zone (SEZ) migrate along the rostral migratory stream (RMS). Once Pax6 is expressed, cells seem to be irreversibly committed to the neuronal lineage.

When Pax6 mRNA levels were analysed, we detected induction of Pax6 3 hours after treatment of cells with anti-CD95 AB (data not shown). Accordingly, at 3 hours after stimulation of CD95, translocation and accumulation of Pax6 in the nucleus was observed (data not shown). Pax6 nuclear accumulation was also found in human neural stem cells 5 hours after stimulation (data not shown). Nuclear Pax6 functions as a transcription factor for proneural genes. These results indicate that CD95L treatment induces expression and nuclear translocation of Pax6, thus conferring commitment of neural stem cells to a neuronal lineage.

Taken together, the results indicate that CD95L is a potent inducer of neurogenesis. Stimulation of CD95 in neural stem cells resulted in loss of stem cell markers, exit from the cell cycle, repression of signalling responsible for stem cell expansion, and induction of neurogenic signalling.

### Example 6

CD95L expression is induced upon injury to the brain, e.g., in stroke. Stroke goes along with proliferation of NSCs that then migrate and integrate into the injured brain region. However nearly all newborn neurons die after 5 weeks. In contrast, inducing targeted apoptosis in selected neurons located in non-neurogenic regions results in proliferation, migration, and integration of adult neural stem cells⁶. Consistently, when E17 neurons are transplanted into the apoptotic cortex they integrate and differentiate into functional mature neurons, but this is not the case if they are transplanted into the necrotic cortex.

To test if endogenous CD95L could induce neurogenesis in vivo, an animal model of global ischemia was used, that is characterized by the fact that only few neurons in the CA1 region of the hippocampus undergo apoptosis while further tissue damage, necrosis and inflammation is minimal. In this model a marked increase in CD95 and CD95L expression in the CA1 region is observed. 48 hours after induction of global ischemia, GFP-expressing neural stem cells derived from the SVZ of wt or lpr mice were injected into the striatum of wt mice. At 7 days after stem cell injection, many GFP positive cells could be detected in the hippocampus in both groups of mice receiving either wt or lpr neural progenitor cells. Analysis of cell numbers in the hilus of the DG and CA1 region did not reveal differences between the groups (data not shown). No differences in numbers of cells double-positive for GFP and the neuronal marker NeuN were detected. GFP⁺/NeuN⁺ cells were barely found in the CA1 regions and hilus of the mice (data not shown).

Notably, at 21 days or 10 weeks after injection strong differences between the two groups of mice in the CA1 region were observed: in mice that received wt cells GFP⁺ cells were detected in the CA1 region. They acquired a neuronal phenotype (NeuN⁺), showed typical neuronal morphology with axons protruding from the cell layer and improved cellularity of the CA1 region (data not shown).

On the contrary, in mice that received lpr cells, we barely found GFP⁺ cells and no GFP⁺/NeuN⁺ positive cells could be detected in the CA1 region (data not shown). When the hilus of the two groups of mice was examined, no significant differences were detected (data not shown), suggesting, that reduction of cell numbers in the CA1 region was not due to poor survival of lpr cells.

### Example 7

A signalling pathway involved in cell survival, migration and neurogenesis is the PI3K pathway. PI3K activates AKT/PKB which can in turn phosphorylate GSK3β thereby inactivating it. This allows β-catenin to escape degradation and act as a transcription factor in the nucleus. To test if PI3K signaling could be responsible for the observed changes in gene expression and translocation of β-catenin to the nucleus we determined phosphorylation of GSK3β and AKT. Stimulation of CD95 resulted in phosphorylation and thus activation of AKT (data not shown). Phosphorylation of AKT was only detected in a close dose range. Doses above the activation dose did not result in phosphorylation of AKT thus describing a bell-shaped activation kinetics. Phosphorylation of AKT exhibited two waves with peaks at 5 min and 15 min. Also phosphorylation of its downstream target GSK3β at the serine 9 residue was detected. Cells expressing a dominant active form of GSK3β did not increase β-III-tubulin expression upon CD95L-T4 treatment (4.60%±0.25 versus 4.79%±0.19). This result indicates, that phosphorylation of GSK313 is necessary for the induction of neuronal differentiation through CD95L.

Phosphorylation of GSK3β could be blocked by treating the cells with the PI3K inhibitor LY 294002. Accordingly PI3K activity was increased at 1 and 5 min after treatment of cells with 1 U of CD95L-T4 (1 versus 1.38±0.12 and 1 versus 1.53±0.18). As CD95 is not a receptor tyrosine kinase, a good candidate connecting CD95 to PI3K could be a non-receptor tryrosine kinase of the Src family kinases. To test if CD95-induced activation of PI3K could be mediated by one of the adaptor molecules of the Src family, we used the Src-family kinase inhibitor PP2. Treatment of NSCs with PP2 inhibited phosphorylation of GSK36 and AKT induced by CD95L.

### Example 8

To further investigate, how CD95 could activate PI3K signalling we performed co-immunoprecipitation experiments (data not shown). With an antibody against CD95 we could co-immunoprecipitate the Src family member pp60 as well as the p85 subunit of PI3K. pp60 and p85 association to CD95 increased at 1 minutes and 5 minutes after stimulation of NSCs with 1 U CD95L-T4. pp60-src is activated by phosphorylation of a tyrosine residue in the activation loop (Y416). We therefore performed immunoprecipitation experiments with an antibody against phosphorylated tyrosine residues. Immunoprecipitates were then probed with an antibody that specifically recognizes Y416. An increase in phosphorylated and thereby active src was observed 5 min after stimulation with CD95L-T4. Using a 1000 fold higher dose of CD95L-T4 (5 μg/ml) there was no increase in phosphorylated src, indicating, that CD95L non-apoptotic signalling acts in a small dose range. To investigate if pp60-src is required for the activation of PI3K signaling and induction of neuronal differentiation NSCs were transfected with siRNA against pp60-src. At 48 h efficient knockdown was confirmed by Western Blot and FACS analysis. In cells containing only the transfection reagent or transfected with control siRNA, stimulation with CD95L-T4 resulted in an increased number of β-III-tubulin positive cells (4.89%±1.61 versus 17.15%±0.65 and 4.69%±0.16 versus 18.07%±4.09 respectively). This increase was blocked in cells transfected with pp60-src siRNA (4.31%±0.43 versus 5.23%±0.79), indicating, that pp60-src is required for the neuronal differentiation phenotype observed after stimulation of CD95.

### Example 9

As we could not detect apoptosis when NSCs were stimulated with CD95L we asked if DISC components were efficiently recruited in these cells. In the adult brain, FADD recruitment to CD95 could readily be detected after 90 min of middle cerebral artery occlusion and 6 hours reperfusion in the ischemic hemisphere (data not shown). When CD95 was stimulated on primary E15 neurons or NSCs we could detect no efficient recruitment of FADD. This result indicates that in immature developing systems like E15 neurons or NSCs stimulation of CD95 does not result in efficient FADD recruitment. Along this line, we did not find cleavage of the executioner Caspase-3 when CD95 was stimulated on NSCs. Furthermore, we could barely detect cells positive for active caspases when NSCs were stimulated with CD95L-T4 or an antibody against CD95.

None of these cells had integrated in the CA1 molecular layer and we did not detect cells that exhibited double positivity for GFP and NeuN. To exclude that this observation was due to poor survival of lpr cells, we examined the hilus of the dentate gyrus of the two groups of mice. In both groups of mice, GFP positive cells had integrated in the dentate gyrus at 10 weeks after injury at a similar extent, indicating that reduction of cell numbers in the CA1 region was not due to poor survival of lpr cells. Taken together, these findings demonstrate that a CD95 signal induces neuronal commitment and further maturation of newly generated NSCs for brain repair.

### Example 10

To test whether transplanted NSCs were functional we asked if they could improve behavioural deficits associated with hippocampal lesions. When placed in a T-maze rats or mice alternate between arms significantly above chance. This spontaneous alternation is decreased in animals with hippocampal lesions. At 5 weeks after ischemia we found, that ischemic mice showed significantly reduced success rates compared to naïve controls (55.76%±3.9 versus 66.44%±4.58). This defect in spontaneous alternation behaviour was rescued in mice injected with wt NSCs, indicating that wt NSCs are able to integrate into the brain circuitry and improve ischemia-induced deficits (64.42%±2.77). In contrast, mice that received lpr NSCs exhibited significantly decreased spontaneous alternation rates comparable to ischemic animals with injection of saline (50.96%±3.3). Apparently, lpr NSCs were not able to integrate into the damaged CA1 and thus were not able to ameliorate behavioural deficits. When given a food reward in additional sessions all groups of animals were able to learn the task in a similar manner. We further tested the exploratory behaviour and spatial recognition of these animals in the modified open field test There were no significant differences between groups in the motor activity in the open field and object exploration, indicating that the effects seen in the spontaneous alternation were not due to changed motor activity or exploratory behaviour in the different groups of mice. When the displaced or exchanged object recognition of mice was analysed, a trend could be observed: mice that had received lpr NSCs spent less time exploring the objects that had been displaced or exchanged compared to mice that had received wt NSCs or control animals, indicating a reduction in nonassociative spatial memory. Taken together, we found that mice treated with lpr NSCs had deficits in spontaneous behaviour as observed in the T-maze and open-field test, while learning tasks as seen in the T-maze and Y-maze (data not shown) were not impaired. These deficits were rescued in mice that received wt NSCs, indicating, that CD95 is required for generation, integration and function of newly born neurons from NSCs.

### DISCUSSION

Several studies illustrate the ability of NSCs to generate new neurons in the SVZ and the hippocampal DG regions in vivo²⁷. First evidence of generation of new neurons by NSCs outside these two neurogenic regions was provided by Magavi et al⁶. They induced selective apoptotic degeneration of cortical neurons by singlet oxygen production. This apoptotic death induced neuronal differentiation of transplanted and endogenous neural progenitors. When E17 neurons are transplanted into the apoptotic cortex they integrate and differentiate into functional mature neurons, but this is not the case if they are transplanted into the necrotic cortex. Consistently, in a model of middle cerebral artery occlusion, characterized by massive cell death with both necrosis and apoptosis, nearly all newborn neurons die after 5 weeks. Magavi et al. speculated that apoptosis induces expression of factors that control neuronal differentiation such as BDNF, NT-3 and NT-4/-5¹¹ or releases, as the authors state, “a repressed program of neural plasticity that permits and/or instructs early neocortical differentiation”. Yet the singlet oxygen production leading to apoptosis is also reported to induce expression of CD95L. Thus, our data add a new molecule to the list of candidate apoptosis-related neurogenic signals.

These observations demonstrate that neuronal fate determination and subtype specification is controlled by signals provided by the microenvironment—the niche. In the neurogenic niche of the dentate gyrus, Wnt signaling has recently been identified as a key regulator guiding adult neurogenesis³¹. Consistently, lack of CD95 did not hamper homing and integration of transplanted NSCs into the DG. By contrast, our data demonstrate that the CD95 system is essential for injury-induced neurogenesis.

We have shown that CD95 ligation results in activation of PI3K signaling and translocation of β-catenin to the nucleus followed by proneural gene expression. There are several observations that support a major role for PI3K signaling in the injury niche: Stroke increases phosphorylation of AKT in NSCs which can be blocked by the PI3K inhibitor LY294002. Further, inhibition of PI3K signaling hinders migration of neuroblasts to the site of injury in explant cultures. Along this line, angiogenesis and neurogenesis that are activated by pharmacological treatment e.g. EPO or statins are mediated by PI3K signaling. Our results now establish the CD95 system as a major regulator of PI3K signaling within the “injury niche”. Interestingly, both Wnt-signaling and PI3K-signaling converge in the inactivation of GSK3β. Yet PI3K blocks GSK3β activity by phosphorylating its Ser 9 and Wnt not. Whether these different means of GSK3β inhibition are responsible for different neuronal fate and subtype specification remain subject of future studies.

We propose that CD95 signals neurogenesis through pp60-Src and PI3K. In the past, several reports have pointed out an important role for tyrosine phosphorylation in CD95 induced signalling. These preliminary reports however suggested that CD95-induced tyrosine phosphorylation is a prerequisite for CD95 mediated apoptosis. Along this line, the phosphatases SHP-1, SHP-2 and SHIP were found to associate through the src-homology domain 2 (SH2) to a phosphotyrosine-containing motif in the DD of CD95 to counteract survival factors-initiated pathways. We now describe a novel association of pp60 and p85 to CD95. Whether another adaptor molecule is still missing in the PI3K-activation complex of CD95 is unclear. Alternatively, pp60 and P85 might directly interact with CD95 through the previously identified phosphotyrosine-containing motif in the DD of CD95 and sequentially activate each other.

Yet, if CD95L induces neurogenesis and is upregulated in the diseased brain, why is neurogenesis after injury in most cases not more pronounced? A possible explanation could be that at the time where CD95L has its highest expression—the acute injury phase—apoptosis is in most cases associated with massive induction of necrosis, inflammation and production of reactive oxygen species. Inflammation is known to compromise neurogenesis: Inducing inflammation by infusion of bacterial lipopolysaccharide (LPS) leads to reactive gliosis and results in a decrease of newborn DG cells of about 85%. Also, in NSCs reactive oxygen species (ROS) seem to be a means to intricately regulate cellular functions, linking cell density to proliferative status. On the other hand, especially NSCs are highly sensitive when it comes to elevated levels of ROS accompanying irradiation-injury: both in vivo and in vitro studies have shown that irradiation depletes NSCs by inducing elevated ROS in these cells.

Following stroke or spinal cord injury, CD95 and CD95L are upregulated leading to cell death. Inhibiting CD95L prevents cells from undergoing apoptosis after injury and results in recovery of motor function in case of spinal cord injury or increased survival after ischemia. Injury to the CNS goes along with proliferation of NSCs. NSCs express the CD95 receptor but as reported here and already by others, they are resistant to CD95-mediated apoptosis. What makes the CD95 signal apoptosis in mature injured neurons and PI3K activation in NSCs? NSCs might lack adaptor molecules required to induce the “death inducing signaling complex” (DISC). Indeed, treatment with CD95L did not induce efficient recruitment of the adaptor molecule FADD in NSCs or developing neurons. In fully differentiated neurons however, FADD was recruited to CD95 following ischemia.

Here we have demonstrated that following global ischemia CD95L drives neurogenesis leading to recovery of ischemia-induced behavioral deficits. CD95L can kill damaged neurons, and promote their replacement by neural progenitor cells and, even support further maturation of these newly generated neurons. Following global ischemia, however, the number of newly generated endogenous NSCs seems to be the limiting factor. Consistently, EGF and bFGF-induced expansion of NSCs resulted in partial recovery of ischemia-induced spatial learning deficits. In contrast, acute neurological insults such as stroke and trauma are accompanied by severe destruction of surrounding tissue, involving necrosis and apoptosis but also a significant expansion of NSCs, most of which fail to become a neuron. Unraveling the factors blocking CD95-mediated neurogenesis in the presence of inflammation, necrosis and ROS will enable the use of CD95 to harness endogenously generated NSCs to repair the injured CNS. Alternatively, over-expression of CD95L in the assumed more permissive environment of the sub-acute phase of ischemic or traumatic CNS injury or neurodegenerative diseases might counteract neuronal loss by increasing neurogenesis from present NSCs. In this case, it is noteworthy that in the adult CNS CD95 expression is only detected in the acute phase of injury. Otherwise there is no detectable expression of CD95 and no risk for mature neurons to undergo CD95-mediated apoptosis. Therefore overexpression strategies can be undertaken in the subacute phase of injury.

Taken together, our data suggest, that apart from being a useful tool for inducing neuronal differentiation of NSCs in vitro, the CD95L system may have an enormous potential for using NSCs to repair the injured CNS.

### CONCLUSION

The data provided herein provides evidence that apoptosis itself might actually control neuronal differentiation. Following injury to the CNS, CD95L kills damaged neurons, and simultaneously promotes their replacement by neural progenitor cells and might even support further maturation of these newly generated neurons.

At the time where CD95L has its highest expression—the acute injury phase—apoptosis is in most cases associated with massive induction of necrosis, inflammation, and production of reactive oxygen species. In this situation, the neurogenic program induced by CD95L might be altered or even absent. Death of neural stem cells following ischemia could therefore not be an active mechanism but simply a consequence of the absence of neurogenic cues.

The data described above suggests that apart from being a useful tool for inducing neuronal differentiation of NSCs in vitro, CD95L or other activators of the CD95L/CD95 system can be used in vivo to endogenously repair the injured CNS.

It will be apparent to those skilled in the art that various modifications and variations can be made in the methods and compositions of the present inventions without departing from the spirit or scope of the invention. Thus, it is intended that the present invention cover the modification and variations of the invention provided they come within the scope of the appended claims and their equivalents.

### REFERENCES

1. Arvidsson, A., Collin, T., Kirik, D., Kokaia, Z. & Lindvall, O, Neuronal replacement from endogenous precursors in the adult brain after stroke. Nat. Med. 8, 963-970 (2002).
2. Emsley, J. G., Mitchell, B. D., Kempermann, G. & Macklis, J. D. Adult neurogenesis and repair of the adult CNS with neural progenitors, precursors, and stem cells. Prog. Neurobiol. 75, 321-341 (2005).
3. Jin, K. et al. Fas (CD95) may mediate delayed cell death in hippocampal CA1 sector after global cerebral ischemia. J. Cereb. Blood Flow Metab 21, 1411-1421 (2001).
4. Lois, C. & Alvarez-Buylla, A. Proliferating subventricular zone cells in the adult mammalian forebrain can differentiate into neurons and glia. Proc. Natl. Acad. Sci. U.S. A 90, 2074-2077 (1993).
5. Temple, S. The development of neural stem cells. Nature 414, 112-117 (2001).
6. Magavi, S. S., Leavitt, B. R. & Macklis, J. D. Induction of neurogenesis in the neocortex of adult mice. Nature 405, 951-955 (2000).
7. Parent, J. M., Vexler, Z. S., Gong, C., Derugin, N. & Ferriero, D. M. Rat forebrain neurogenesis and striatal neuron replacement after focal stroke. Ann. Neurol. 52, 802-813 (2002).
8. Demjen, D. et al. Neutralization of CD95 ligand promotes regeneration and functional recovery after spinal cord injury. Nat. Med. 10, 389-395 (2004).
9. Martin-Villalba, A. et al. CD95 ligand (Fas-L/APO-1L) and tumor necrosis factor-related apoptosis—inducing ligand mediate ischemia-induced apoptosis in neurons. J Neurosci 19, 3809-17 (1999).
10. Matsushita, K. et al. Fas receptor and neuronal cell death after spinal cord ischemia. J Neurosci 20, 6879-87 (2000).
11. Wang, Y., Sheen, V. L. & Macklis, J. D. Cortical interneurons upregulate neurotrophins in vivo in response to targeted apoptotic degeneration of neighboring pyramidal neurons. Exp. Neurol. 154, 389-402 (1998).
12. Krammer, P. H. CD95's deadly mission in the immune system. Nature 407, 789-795 (2000).
13. Ricci-Vitiani, L. et al. Absence of caspase 8 and high expression of PED protect primitive neural cells from cell death. J. Exp. Med. 200, 1257-1266 (2004).
14. Tamm, C. et al. Differential regulation of the mitochondrial and death receptor pathways in neural stem cells. Eur. J. Neurosci. 19, 2613-2621 (2004).
15. Zuliani, C. et al. Control of neuronal branching by the death receptor CD95 (Fas/Apo-1). Cell Death. Differ. 13, 31-40 (2006).
16. Desbarats, J. et al. Fas engagement induces neurite growth through ERK activation and p35 upregulation. Nat Cell Biol 5, 118-25 (2003).
17. Landau, A. M. et al. Defective Fas expression exacerbates neurotoxicity in a model of Parkinson's disease. J. Exp. Med. 202, 575-581 (2005).
18. Jacobs, S. et al. Retinoic acid is required early during adult neurogenesis in the dentate gyrus. Proc. Natl. Acad. Sci. U.S. A 103, 3902-3907 (2006).
19. Hermanson, O., Jepsen, K. & Rosenfeld, M. G. N-CoR controls differentiation of neural stem cells into astrocytes. Nature 419, 934-939 (2002).
20. Adachi, M., Watanabe-Fukunaga, R. & Nagata, S. Aberrant transcription caused by the insertion of an early transposable element in an intron of the Fas antigen gene of lpr mice. Proc Natl Acad Sci USA 90, 1756-60 (1993).
21. Lendahl, U., Zimmerman, L. B. & McKay, R. D. CNS stem cells express a new class of intermediate filament protein. Cell 60, 585-595 (1990).
22. Hunter, T. & Pines, J. Cyclins and cancer. Cell 66, 1071-1074 (1991).
23. Androutsellis-Theotokis, A. et al. Notch signalling regulates stem cell numbers in vitro and in vivo. Nature (2006).
24. Sun, Y. et al. Neurogenin promotes neurogenesis and inhibits glial differentiation by independent mechanisms. Cell 104, 365-376 (2001).
25. Shu, T. et al. Doublecortin-like kinase controls neurogenesis by regulating mitotic spindles and M phase progression. Neuron 49, 25-39 (2006).
26. Takahashi, J., Palmer, T. D. & Gage, F. H. Retinoic acid and neurotrophins collaborate to regulate neurogenesis in adult-derived neural stem cell cultures. J. Neurobiol. 38, 65-81 (1999).
27. Hack, M. A. et al. Neuronal fate determinants of adult olfactory bulb neurogenesis. Nat. Neurosci. 8, 865-872 (2005).
28. Martin-Villalba, A. et al. Therapeutic neutralization of CD95-ligand and TNF attenuates brain damage in stroke. Cell Death Differ 8, 679-86 (2001).
29. Sheen, V. L. & Macklis, J. D. Targeted neocortical cell death in adult mice guides migration and differentiation of transplanted embryonic neurons. J. Neurosci. 15, 8378-8392 (1995).
30. Ray J, Peterson D A, Schinstine M, & Gage F H. Proliferation, differentiation, and long-term culture of primary hippocampal neurons. Proc. Natl. Acad. Sci. U.S.A.; 90:3602-3606. (1993).
31. Cha, S. S. et al. Crystal structure of TRAIL-DR5 complex identifies a critical role of the unique frame insertion in conferring recognition specificity. J. Biol. Chem. 275, 31171-31177 (2000).
32. Eck, M. J. & Sprang, S. R. The structure of tumor necrosis factor-alpha at 2.6 A resolution. Implications for receptor binding. J Biol Chem 264, 17595-605 (1989).
33. Hymowitz, S. G. et al., Triggering cell death: the crystal structure of Apo2L/TRAIL in a complex with death receptor 5. Mol Cell 4, 563-71 (1999).
34. Guthe, S. et al. Very fast folding and association of a trimerization domain from bacteriophage T4 fibritin. J. Mol. Biol. 337, 905-915 (2004).
35. Meier, S., Guthe, S., Kiefhaber, T., & Grzesiek, S. Foldon, the natural trimerization domain of T4 fibritin, dissociates into a monomeric A-state form containing a stable beta-hairpin: atomic details of trimer dissociation and local beta-hairpin stability from residual dipolar couplings. J. Mol. Biol. 344, 1051-1069 (2004).

1. Use of a composition comprising an activator of the CD95-Ligand (CD95L)/CD95-Receptor (CD95) system for the manufacture of a medicament for the treatment of a neuronal injury.

2. The use of claim 1, wherein the activator is selected from
(i) CD 95L₁ and
(ii) an activating antibody directed against the CD95 receptor.

3. The use of claim 1, wherein the neuronal injury is a neurodegenerative disease such as Parkinson's disease, Alzheimer's disease.

4. The use of claim 1, wherein the neuronal injury is stroke or spinal cord injury.

5. The use of claim 1, wherein the treatment is in a subacute stage of the disease or injury.

6. The use of claim 1, wherein the treatment is for inducing neuronal differentiation in vitro or in vivo.

7. The use of claim 1, wherein the treatment comprises
(i) an administration of the activator to neuronal stem cells, and
(ii) introduction or migration of the treated stem cells to the site of neuronal disease or injury.

8. The use of a composition comprising a polynucleotide encoding CD95L for the manufacture of a medicament for the treatment of a neuronal disease or a neuronal injury.

9. The use of claim 8, wherein the treatment comprises:
(i) an administration of the polynucleotide to neuronal stem cells and
(ii) introduction or migration of the treated stem cells to the site of neuronal disease or injury.

10. A method for treating injured neuronal tissue comprising:
(i) treating stem cells with an activator of the CD95-Ligand (CD95L), CD95-Receptor (CD95) system, and
(ii) introducing those treated stem cells to the site of neuronal injury, wherein the treated stem cells differentiate into new neuronal cells at the site of injury.

11. The method of claim 10, wherein the activator is CD95L or an activator antibody against CD95.

12. The method of claim 10, wherein the neuronal tissue is mammalian neuronal tissue.

13. The method of claim 10, wherein the mammal is a human patient who has Parkinson's disease, or has suffered from a neuronal injury, such as stroke or spinal cord injury.

14. The method of claim 12, wherein the injured neuronal tissue is in the brain of the mammal or elsewhere in the mammal's central nervous system.

15. A method for treating injured neuronal tissue comprising:
(i) engineering a stem cell to express a polynucleotide encoding CD95L; and
(ii) introducing that engineered stem cell into a subject, wherein:
(i) the stem cell migrates to the site of injury; and
(ii) (ii) the polynucleotide is operably linked to regulatory elements that are preferably only active in the tissue that has been injured.

16. A method for treating injured neuronal tissue comprising introducing a viral vector into a subject, wherein the vector comprises a polynucleotide that encodes CD95L and is operably linked to regulatory elements that are preferably only active in the tissue that has been injured.

17. The method of claim 16, wherein the vector is an adenovirus or lentivirus vector.

18. A vector comprising a polynucleotide that encodes a CD95L protein, wherein the polynucleotide is operably linked to:
(i) a promoter that is preferably active in neuronal tissue only and
(ii) a terminator functional in neuronal tissue.

19. The vector of claim 18, wherein the CD95L protein comprises the sequence depicted in SEQ ID NO: 3.

20. A stem cell, which comprises in its genome a non-naturally occurring polynucleotide that comprises a polynucleotide that encodes a CD95L protein, wherein the polynucleotide is operably linked to:
(i) a promoter that is preferably active in neuronal tissue only, and
(ii) a terminator functional in neuronal tissue.
